(19) United States
(12) Patent Application Publication
(10) Pub. No.: US 2012/0209131 A1
(43) Pub. Date: Aug. 16, 2012

(54) Method and System of a Cardio-acoustic Classification system for Screening, Diagnosis and Monitoring of Cardiovascular Conditions

(75) Inventors: Kevin Jones, Gainesville, FL (US); Garima Srivastava, Sunrise, FL (US)

(73) Assignee: AventuSoft, LLC, Sunrise, FL (US)

(21) Appl. No.: 13/370,514

(22) Filed: Feb. 10, 2012

Related U.S. Application Data
(60) Provisional application No. 61/463,092, filed on Feb. 11, 2011.

(51) Int. Cl. A61B 5/02; A61B 7/00

(52) U.S. Cl. 600/528

(57) ABSTRACT

A method and system are provided for a portable cardio-acoustic device. The device includes a display with user input, a sensor array to capture heart related vibrations from infrasound and acoustically transmitted audible sound, and a processor to extract salient features in accordance with human factor analysis, separate heart sounds as a function of sound patterns modeled from mechanical and physiological processes of the heart, classify heart sound patterns in accordance with biologically based signal processing models of the auditory cortex and cerebellum, and diagnose and monitor cardiovascular condition based on the classification of the heart sound patterns.

### CROSS REFERENCE TO RELATED APPLICATION

This application also claims priority benefit to Provisional Patent Application No. 61/463,092 filed on Feb. 11, 2011, the entire contents of which are hereby incorporated by reference.

### FIELD OF THE INVENTION

The embodiments herein relate generally to health monitoring and more particularly to sound analysis software and acoustic listening devices in medical practice.

### BACKGROUND

Auscultation is the process of listening to internal body sounds, and includes, for example, the technique of listening to heart sounds for the diagnosis of heart disorders using a stethoscope. Cardiologists perform accurate diagnosis using auscultation, but accurate diagnosis using the auscultation technique is problematic for pediatricians, internists, primary care physicians, physician assistants, registered nurses, nurse practitioners and other non-cardiologist healthcare professionals.

Non-cardiologist healthcare professionals are prone to inaccurate diagnoses during cardiovascular examinations. It is further reported that a large percentage of medical graduates cannot properly diagnose heart conditions using a stethoscope. Inaccurate diagnoses lead to premature referrals for cardiologist consultations and expensive medical procedures such as two-dimensional echocardiograms (2-D echo). Extensive procedures that result in benign auscultatory findings and failure to recognize abnormal cardiac function during examinations are two significant problems of current medical practice.

A need therefore exists for listening devices that assist the clinician in their practice for making a proper diagnosis.

### BRIEF DESCRIPTION OF THE DRAWINGS

The features of the system, which are believed to be novel, are set forth with particularity in the appended claims. The embodiments herein, can be understood by reference to the following description, taken in conjunction with the accompanying drawings, in the several figures of which like reference numerals identify like elements, and in which:

FIG. 1A illustrates a device for cardio-acoustic classification, screening, diagnosis and monitoring of cardiovascular condition;

FIG. 1B illustrates a communication network for monitoring and reporting cardiovascular condition in accordance with one embodiment;

FIG. 2 illustrates a portable cardio-acoustic device to perform classification, screening, diagnosis and monitoring of cardiovascular condition in accordance with one embodiment;

FIG. 3 illustrates an exemplary audibility curve of human sensitivity in accordance with one embodiment;

FIG. 4 presents a method for providing, monitoring and managing cardiovascular condition by way of the portable cardio-acoustic device in accordance with one embodiment;

FIG. 5 illustrates an exemplary cardiac signal (heart sounds) in accordance with one embodiment;

FIG. 6 illustrates an exemplary human factor filter-bank suitable for use with cardiac signals in accordance with one embodiment; and

FIG. 7 illustrates a Time-Frequency analysis of heart sounds in accordance in accordance with one embodiment.

FIG. 8. illustrates the contact sensor's (microphones) to capture infrasonic and audible sounds.

### DETAILED DESCRIPTION

While the specification concludes with claims defining the features of the embodiments of the invention that are regarded as novel, it is believed that the method, system, and other embodiments will be better understood from a consideration of the following description in conjunction with the drawing figures, in which like reference numerals are carried forward.

As required, detailed embodiments of the present method and system are disclosed herein. However, it is to be understood that the disclosed embodiments are merely exemplary, which can be embodied in various forms. Therefore, specific structural and functional details disclosed herein are not to be interpreted as limiting, but merely as a basis for the claims and as a representative basis for teaching one skilled in the art to variously employ the embodiments of the present invention in virtually any appropriately detailed structure. Further, the terms and phrases used herein are not intended to be limiting but rather to provide an understandable description of the embodiment herein.

Briefly, the terms “a” or “an,” as used herein, are defined as one or more than one. The term “plurality,” as used herein, is defined as two or more than two. The term “another,” as used herein, is defined as at least a second or more. The terms “including” and/or “having,” as used herein, are defined as comprising (i.e., open language). The term “coupled,” as used herein, is defined as connected, although not necessarily directly, and not necessarily mechanically. The term “suppressing” can be defined as reducing or removing, either partially or completely. The term “processing” can be defined as number of suitable processors, controllers, units, or the like that carry out a pre-programmed or programmed set of instructions.

The terms “program,” “software application,” and the like as used herein, are defined as a sequence of instructions designed for execution on a computer system. A program, computer program, or software application may include a subroutine, a function, a procedure, an object method, an object implementation, an executable application, an applet, a servlet, a source code, an object code, a shared library/dynamic load library and/or other sequence of instructions designed for execution on a computer system.

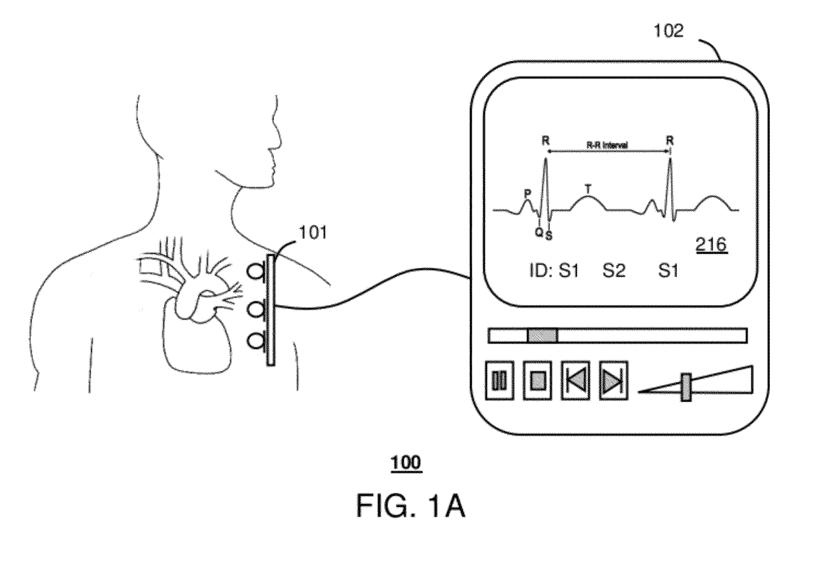

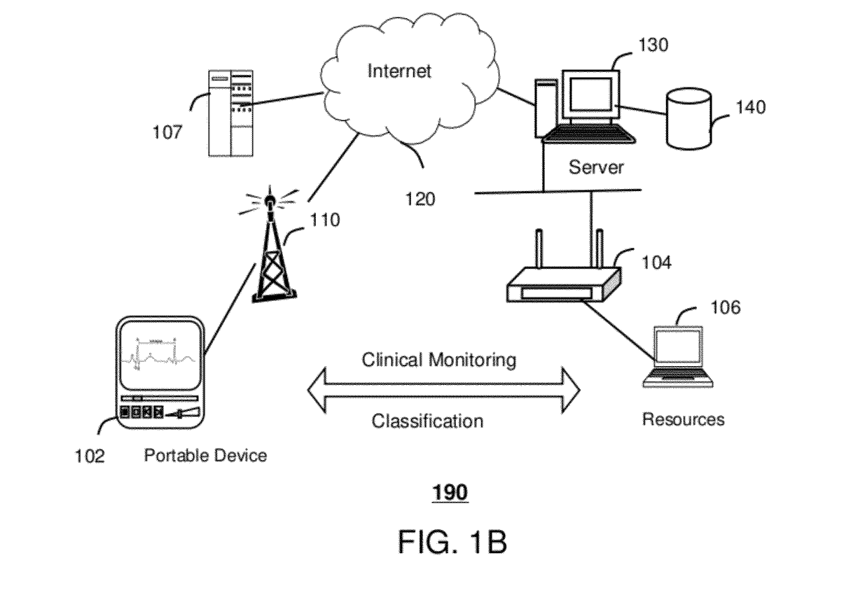

Referring to FIG. 1A, an illustration 100 for performing auscultation by way of the inventive listening device, and methods herein described, is provided. As shown, the portable cardio-acoustic device 102 within a clinical setting performs cardio-acoustic classification, screening, diagnosis and monitoring of cardiovascular condition. The sensor array 101 communicatively coupled thereto listens to heart sounds that are provided to the portable cardio-acoustic device 102 for processing and analysis. Use of the portable device 102, does not require specialized training, is non-invasive, and allows non-cardiologists to provide adequate diagnostic capabilities for detecting normal and abnormal conditions of the cardiovascular system. It is capable of working with both congenital and acquired heart diseases.

In a first embodiment, the exemplary portable cardio-acoustic device 102 and method of operation herein provides for Human-Factor Cardio-acoustic Classification System (HFCCS), to automatically classify cardiovascular diseases from the captured heart sounds. Specifically, psychoacoustics is considered in classifying cardiovascular diseases from the heart sound signal captured through the sensory array 101, including the infrasound regions of the heart sounds. These are sounds lower than the human audible range. The embedded platform, the psychoacoustics, and a related method are discussed in further detail ahead.

In a second embodiment, the exemplary portable cardio-acoustic device 102 and method of operation herein provide a novel approach for providing classification of heart murmurs. Specifically, the heart sounds are separated as a function of heart sound patterns and knowledge of psychoacoustics. Using this further information, conventional issues are bypassed allowing measurements to be carried out from heart sounds. Conventional noise cancellation techniques also have poor performance working with heart sounds. The use of the heart sound pattern, the signal from infrasound region and novel psychoacoustics avoid these issues altogether, as will be discussed ahead.

Referring to FIG. 1B, a portable communication environment 190 is shown for monitoring and reporting a health status responsive to the classification of the heart sounds by the portable cardio-acoustic device 102. The environment 190 enables the portable cardio-acoustic device to monitor and report a health status locally to the immediate user, or to a remote resource, responsive to the classification of the heart sounds. Through this communication network, the portable cardio-acoustic device can monitor and report a health status responsive to the classification of the heart sounds. As one example, it can detect and report normal and abnormal conditions of the cardiovascular system for both congenital and acquired heart diseases.

The portable communication environment 190 can provide wireless connectivity over a radio frequency (RF) communication network, a Wireless Local Area Network (WLAN) or other telecom, circuit switched, packet switched, message based or network communication system. In one arrangement, the portable device 102 can communicate with a base receiver 110 using a standard communication protocol such as CDMA, GSM, TDMA, etc. The base receiver 110, in turn, can connect the portable device 102 to the Internet 120 over a packet switched link. The internet can support application services and service layers 107 for providing media or content to the portable device 102. The portable device 102 can also connect to other communication devices through the Internet 120 using a wireless communication channel. The portable device 102 can establish connections with a server 130 on the network and with other portable devices for exchanging information. The server 130 can have access to a database 140 that is stored locally or remotely and which can contain profile data. The server can also host application services directly, or over the internet 120. In one arrangement, the server 130 can be an information server for entering and retrieving presence data.

The portable device 102 can also connect to the Internet over a WLAN 104. Wireless Local Access Networks (WLANs) provide wireless access to the portable communication environment 190 within a local geographical area. WLANs can also complement loading on a cellular system, so as to increase capacity. WLANs are typically composed of a cluster of Access Points (APs) 104 also known as base stations. The portable communication device 102 can communicate with other WLAN stations such as laptops within the base station area. In typical WLAN implementations, the physical layer uses a variety of technologies such as 802.11 a/b/g/n technologies. The portable device 102 can send and receive data to the server 130 or other remote servers on the portable communication environment 100. In one example, the portable device 102 can send and receive images from the database 140 through the server 130.

As one networked systems example, the system for assessing cardiovascular condition can include a portable cardio-acoustic device 102 and a remote server 130 communicatively coupled to the portable cardio-acoustic device 102. The device can comprise a display with user input; a sensor array to capture from heart sounds, both vibrations from infrasound and acoustically transmitted audible sound; a processor coupled to the display and sensor array to extract salient features from a captured heart sounds in accordance with human factor analysis and separate heart sounds as a function of sound patterns modeled from mechanical and physiological processes of the heart determined through phsychoacoustic analysis. The server 130 can classify heart sound patterns in accordance with biologically based signal processing models of the auditory cortex and cerebellum; and diagnose and monitor cardiovascular condition based on the classification of the heart sound patterns. The remote server can compare sound patters against pre-determined models of the mechanical and physiological processes of heart sounds stored in a local database 140 determined through phsychoacoustic analysis; and classify if a murmur detected in the heart sounds from the sound patternsis innocent or pathological and a type of innocent murmur, wherein the pre-stored models are derived from phsychoacoustic analysis of known regular and irregular heart sounds, and including identifying the type of innocent murmur and type of pathological murmur or other cardiovascular classification. The remote server 140 can respond to a software application executing on a mobile device 102 requesting classification of the sound patterns and visually presents a psychoacoustic interpretation of sound patterns, for on-line rendering or on the mobile device.

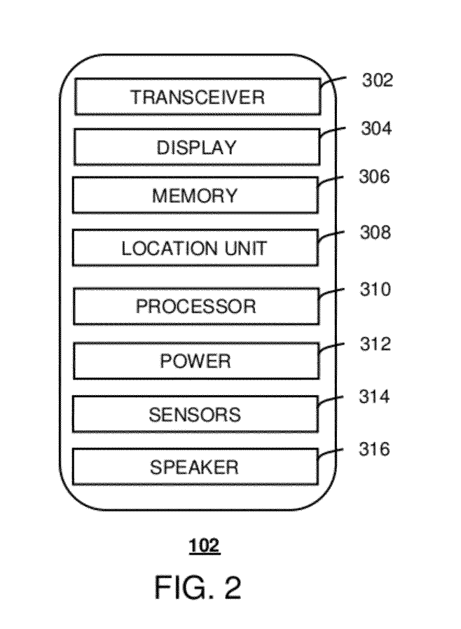

FIG. 2 depicts an exemplary embodiment of the portable device 102 for performing cardio-acoustic classification, screening, diagnosis and monitoring of cardiovascular condition. It comprises a wired and/or wireless transceiver 302, a user interface (UI) display 304, a memory 306, a location unit 308, and a processor 310 for managing operations thereof. The portable device 102 can be any smart processing platform with Digital signal processing capabilities, application processor, data storage, display, input modality like touch-screen or keypad, microphones, speaker, Bluetooth, and connection to the internet via WAN, Wi-Fi, Ethernet or USB. This embodies custom hardware devices, smartphone, cell phone, mobile device, iPad and iPod like devices, a laptop, a notebook, a tablet, or any other type of portable and mobile communication device. A power supply 312 provides energy for electronic components.

In one embodiment where the portable device 102 operates in a landline environment, the transceiver 302 can utilize common wire-line access technology to support POTS or VoIP services. In a wireless communications setting, the transceiver 302 can utilize common technologies to support singly or in combination any number of wireless access technologies including without limitation cordless phone technology (e.g., DECT), Bluetooth™, Wireless Fidelity (WiFi), Worldwide Interoperability for Microwave Access (WiMAX), Ultra Wide Band (UWB), software defined radio (SDR), and cellular access technologies such as CDMA-1X, W-CDMA/HSDPA, GSM/GPRS, TDMA/EDGE, and EVDO. SDR can be utilized for accessing a public or private communication spectrum according to any number of communication protocols that can be dynamically downloaded over-the-air to the communication device. It should be noted also that next generation wireless access technologies can be applied to the present disclosure.

The power supply 312 can utilize common power management technologies such as power from USB, replaceable batteries, supply regulation technologies, and charging system technologies for supplying energy to the components of the communication device and to facilitate portable applications. In stationary applications, the power supply 312 can be modified so as to extract energy from a common wall outlet and thereby supply DC power to the components of the communication device 102.

The location unit 308 can utilize common technology such as a GPS (Global Positioning System) receiver that can intercept satellite signals and there from determine a location fix of the portable device 102.

The controller processor 310 can utilize computing technologies such as a microprocessor and/or digital signal processor (DSP) with associated storage memory such a Flash, ROM, RAM, SRAM, DRAM or other like technologies for controlling operations of the aforementioned components of the communication device.

The portable device 102 also includes the sensors 314 for capturing heart signals and environmental sounds and a speaker 316 for playing audio or other sound media. One or more microphones may be present as a sensor array for enhanced noise suppression such as adaptive beam canceling, and one or more speakers 316 may be present for stereophonic sound reproduction. The sensors 314 capture heart sounds from the patient's heart, including both:
1) vibrations from infrasound, and
2) acoustically transmitted audible sound.

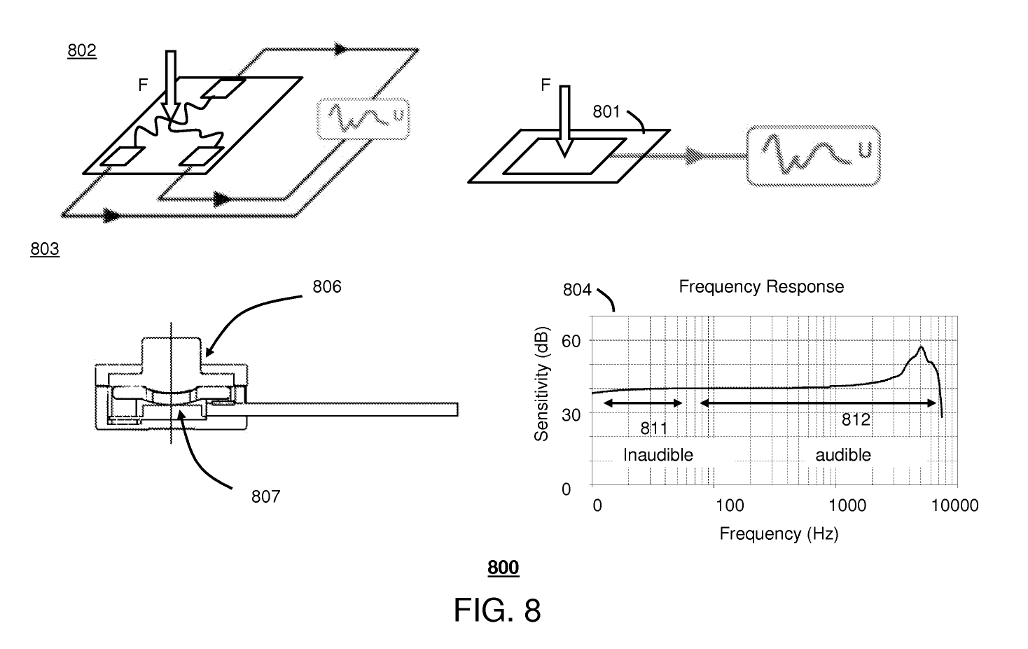

Briefly referring ahead to FIG. 8, an exemplary embodiment 800 of a sensor array 802 for capturing infrasound and audible sound from the heart. The sensor array 802 includes multiple sensors 801 that pick up vibrations from the body and the air. In such combined form, the sensor array is capable of providing a frequency range 804 specific to sensitivity in the infrasound region 811 and also the audible region 812. In one arrangement as shown in 802, the sensors are placed in a non-colinear arrangement such that the position and magnitude of an applied force (F) can be localized; wherein the force is generated in response to a body function, for example, a compression or decompression of chest walls responsive to a heart beat, respiratory function or muscle contraction. The processor evaluates the location of the force over time to generate a body signal vector; a mathematical quantity that has both magnitude and direction. In such arrangement, even though the force may be localized to a single spatial point (e.g., <x,y,z>) as shown in 802 on the surface of the sensor array, the physiological mechanisms which drive the point source that emanate from differing locations can be identified and tracked (e.g., blood flow or muscles of the chambers of the heart: right heart ventricle, left ventricle, right atrium, left atrium.)

In one embodiment, as shown in 803, each sensor 801 includes a front sensing non-contact component 806 and a bottom sensing contact component 807. The contract component 807 can rest on the body surface to detect infrasound body sounds and vibration; direct contact provides a reduction in acoustic impedence to maximizes acoustic sound propagation of infrasound. Lower impedences permit for improved acoustic wave propogation. It comprises a flexible membrane that transforms shape responsive to mechanical forces, which is measured via the sensor. As one example, the membrane is a piezoelectric material that generates an electric voltage responsive to an applied force. The contact sensor also includes an adhesive, which can include a gel paste, to further enhance impedence matching. The non-contact component 806 embodies microphonic elements that are exposed to air to provide for capture of acoustic waveforms. It can react as a micro-electro mechanical microphone, electet or other type of condenser microphone responsive to acoustic waveforms. The sensor array 802 thus shares construct to embody contact sensors for body sounds on a back side and non-contact sensors on a front side for audible sounds, for example, the outer diaphgram support housing, certain electrical wiring traces, and integrated design layout.

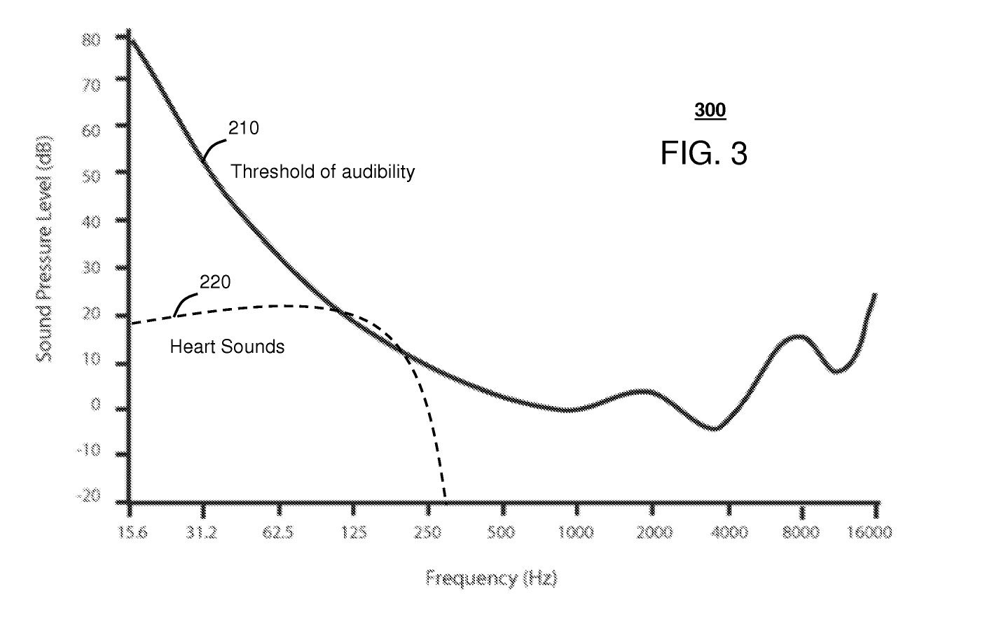

Heart sounds are produced by the vibrations of the cardiohemic system, composed of the blood, heart walls and valves. The vibrations are triggered by the acceleration and deceleration of blood due to abrupt mechanical events of the cardiac cycle. Sounds present at the chest wall are the result of the heart muscles, together with the sound transmission characteristic of the heart and chest wall. A portion of sound produced by these vibrations lies in the human audible frequency range and a portion lies in the lower-frequency inaudible infra-sound range. Heart sounds recorded on the chest wall are found between 0-1000 Hz with the main energy below 100 Hz (FIG. 3: 220).

Briefly, FIG. 3 illustrates a threshold of human audibility curve 210 and the envelope 220 of sound levels produced by heart sounds. The normal hearing range for humans is 20-20,000 Hz. Plot 210 shows the sensitivity of human hearing is amplitude dependent on frequency. Amplitude is the degree of displacement of air molecules whereas loudness is the subjective perception of amplitude by the ear. Plot 210 shows the highest sensitivity between 1000-5000 Hz; that is, sounds within this region are best perceived. The intensity required to hear rises sharply as frequency decreases with inability to hear below 20 Hz. The plot 210 illustrates the threshold of human hearing over frequency and amplitude. For any specific frequency, any sound produced with a Sound Pressure Level (SPL) below the threshold line (plot 210) will not be audible to humans; that is, a person will not hear the sound. The illustration shows that the heart sounds, as illustrated by the envelope 220, are generally below the human audibility level curve 210.

Hearing sensitivity is explained through psychoacoustics, which is the study of sound perception and the relationship between sounds and its physiological and psychological effects. Hearing is not a purely mechanical phenomenon of air and fluid wave propagation, but is also a sensory and perceptual event; in other words, when a person hears something, that something arrives at the ear as a mechanical sound wave traveling through the air, but within the ear it is transformed into neural action potentials through fluid movement across the basilar membrane. Inner hair fibers on the basilar membrane are motioned back and forth responsive to the fluid movement. This mechanical movement generates action potentials due to the opening and closing of hair cell membranes which results in the passage of charged particles, thus generating electro-chemical gradients known as the action potentials. These nerve pulses then travel to the brain where they are perceived through higher level cognition processes. Hence, in many problems in acoustics, such as for audio processing, it is advantageous to take into account not just the mechanics of the environment, but also the fact that both the ear and the brain are involved in a person's listening experience.

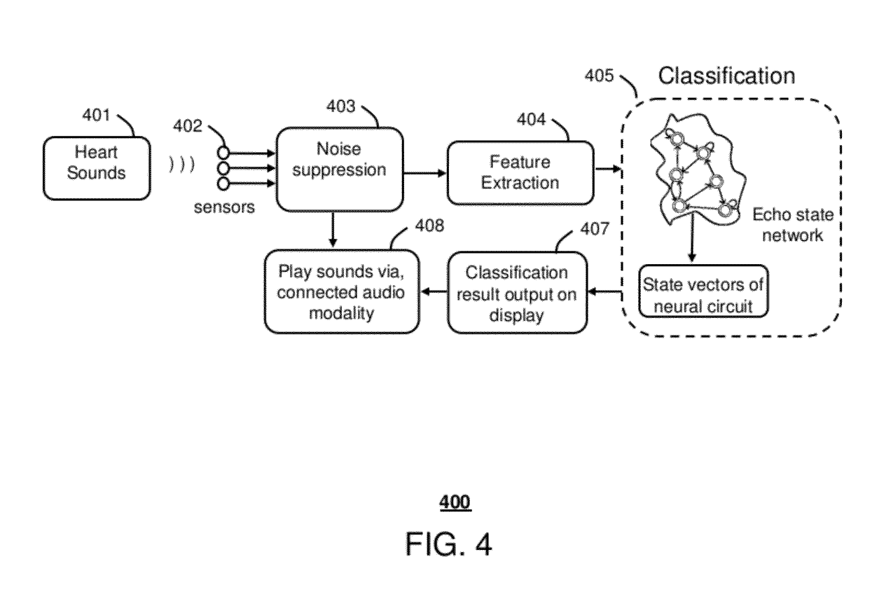

The exemplary embodiments herein provide a device and method that incorporates both the mechanical aspect of hearing physiology and cognitive perception of sound for detecting and classifying heart sounds. Referring now to FIG. 4, a method for performing cardio-acoustic classification, screening, diagnosis and monitoring of cardiovascular condition is herein provided. The method 400 can be provided with more or less than the number of steps shown. When describing the method 400, reference will be made to FIGS. 1, 2, 3 and to 6-8, although it must be noted that the method 400 can be practiced in any other suitable system or device. The steps of the method 400 are not limited to the particular order in which they are presented in FIG. 4. The method can also have a greater number of steps or a fewer number of steps than those shown in FIG. 4.

The method 400 can start in state 401 as shown in FIG. 1, for example, where the sensor array 101 of the portable cardio-acoustic device 102 is placed on the patient chest to listen for heart sounds. The sensor array at state 402, captures from the heart sounds, both vibrations from infrasound and acoustically transmitted audible sound. One or multiple microphones of the sensor array may be piezoelectric based to capture infrasound signals. These microphones 800 consist of piezoelectric film sensor contacts to capture vibrations from infrasound and audible-sound regions with high fidelity. An additional benefit is that they do not capture environmental noise. Captured cardiac sounds can be played immediately on acquisition, as shown in step 408, for example to serve as an enhanced stethoscope for performing auscultation, or after classification, wherein specific identified heart sounds characteristic of irregular heart functions can be played, or looped, for reviewing sound clip specifics. The portable cardio-acoustic device also permits the heart sounds to be played above human hearing threshold as explained ahead.

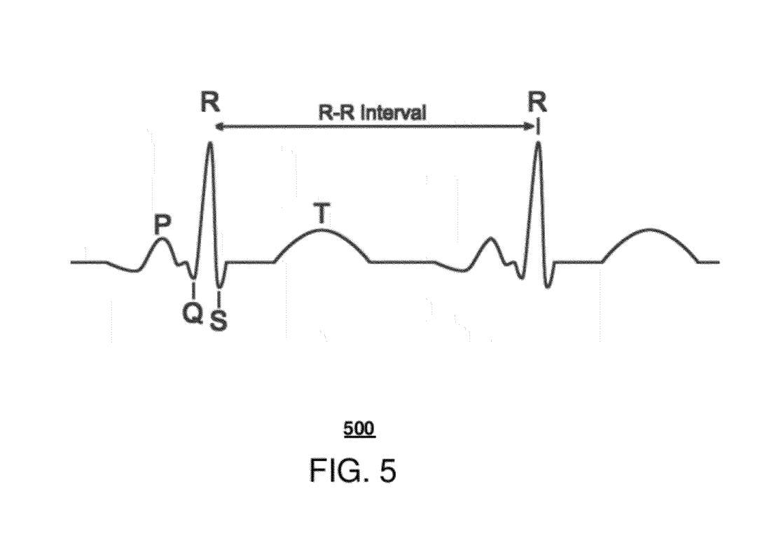

Referring briefly to FIG. 5, an exemplary cardiac signal 500 is illustrated, for instance, one captured by the portable-acoustic device 102. The P wave describes the depolarization of the right and left atria. The amplitude of this wave is relatively small, because the atrial muscle mass is limited. The QRS complex corresponds to the largest wave, since it represents the depolarization of the right and left ventricles, being the heart chambers with substantial mass. Finally, the Twave depicts the ventricular repolarization. It has a smaller amplitude, compared to the QRS complex. However, its precise position depends on the heart rate, e.g., appearing closer to the QRS waves at rapid heart rates. The heart beat can be estimated by measuring the R-R interval of the signal.

Heartbeats vary depending on various factors such as age, physical state, and stimuli. People with heart disease produce irregular sounds that are emitted for each heartbeat. Their blood flows through abnormal valves causing murmurs—an important diagnosis for cardiac diseases. Frequency is denoted as the number of times a regularly recurring phenomenon occurs in one second. A sound with a low frequency will have a low pitch, as generated by a human's heartbeat. Some low frequencies generated by the heart beat below 20 Hz that cannot be heard, can be detected by the portable cardio-acoustic device 102.

Returning back to FIG. 4, at step 403, noise suppression is applied to the captured heart signal to generate a cardiac signal specific to the psychoacoustics of the heart sounds. In one arrangement, the processor 310 performing the noise suppression non-linearily amplifies the heart sounds in the infrasound bandwidth below hearing threshold in accordance with a psychoacoustic compression to compensate for the biological representation of loudness, pitch and timbre of human hearing to produce the cardiac signal. This can encode masking effects analogous to the manner in which the cochlea masks frequency specific sounds across a human hearing scale. Progressive masking of high frequencies by lower frequencies occurs as amplitude rises, which the noise suppression emulates.

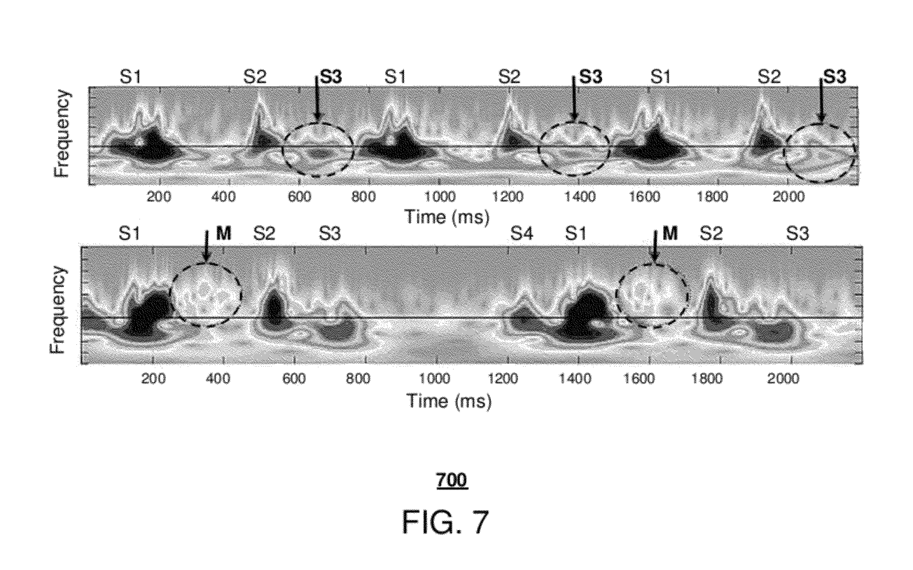

In another arrangement the processor 310 non-linearily frequency shifts the cardiac signal in the infrasound bandwidth to the audible bandwidth in accordance with a human hearing frequency scale (see also FIG. 7) to compensate for the biological representation of loudness, pitch and timbre of human hearing to produce the cardiac signal. This can also encode masking effects analogous to the manner in which the cochlea masks frequency specific sounds across a human hearing scale to permit audibility of the heart sound above human hearing sensitivity threshold.

At step 404 salient features are extracted from the captured cardiac signal of the heart sounds in accordance with human factor analysis. As part of this feature extraction, the processor 310 enhances sensitivity in a low frequency infrasound range of the heart sounds, and identifies variations of the cardiac signal in the infrasound range according to classified heart disease indicators. A portion of the feature extraction includes applying a human factor critical band filter-bank to the infrasound and acoustically transmitted audible sound to increase resolution below 100 Hz and enhance sensitivity to the lower frequency regions specific to the heart sounds. This may be done in conjunction with the feature extraction, or as part of the noise suppression, depending on the desired programming implementation.

The filter bank is derived from the extracted features. The feature extraction technique is inspired by an accurate model of the human auditory system, designed to match the human hearing performance in the 100-10,000 Hz region (see FIG. 7). Recal, Pressure changes in the air reach the ear drum and are transmitted to the cochlea. Pressure waves induce vibrations of the basilar membrane which in turn induce hearing strains of the inner hair cells. Frequency perception is derived from the position of the inner hair cells grouped in critical bands along the length of the cochlea.

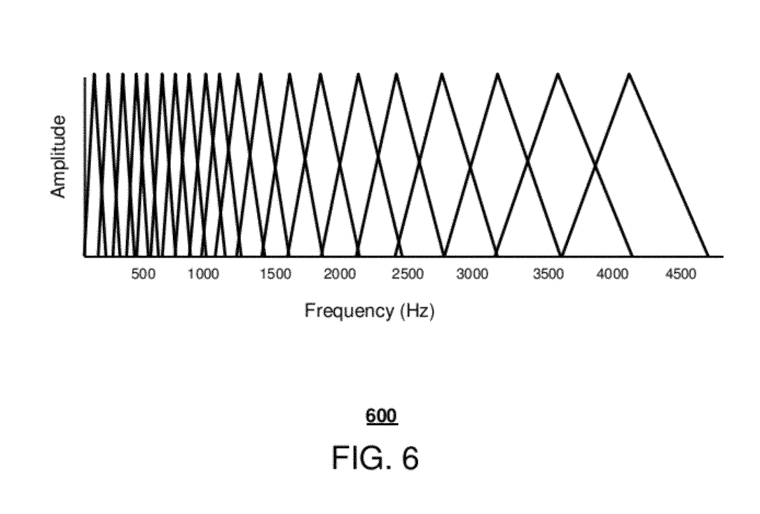

Referring briefly to FIG. 6, an exemplary embodiment of the applied filter bank 600, namely the human-factor-cepstral-coefficient (HFCC) filter bank is shown, is a similar variation to the filter bank constructed from Mel-frequency-cepstral-coefficients. Either may be employed. The HFCC of the inventive design includes frequency scaling and amplitude compression in accordance with the psychoacoustic principles disclosed herein. Recall, hearing or audition is the sense of sound perception and results from tiny hair fibers in the inner ear detecting the motion of atmospheric particles within (at best) a range of 20 to 20000 Hz. As one example, the processor 310 during frequency extraction encodes a time-frequency sound signal decomposition according to frequency perception derived from inner hair cell activation responses grouped in critical bands along the basilar membrane. The human factor critical band filter-bank is one of the steps in the method 400 related to Human Factor Analysis, as will be described ahead.

Human factor analysis addresses the three limiting factors: 1) people have very poor hearing sensitivity in the low frequency infrasound range of the heart sounds. 2) cardiologists are trained and experienced in identifying heart signal variations at these low frequencies through years of extensive clinical training and experience trained for classification of heart disease, and 3) few automated screening device exist today that can reliably and accurately determine the presence of congenital heart disease in an easy-to-use and portable solution, factors that limit the diagnostic capabilities of non-cardiologists. The method 400 provides biologically based model to overcome these limitations, drawing inspiration from the mechanical function of the heart and the human auditory system to model the physiological process of heart sounds, psychoacoustics of hearing and classification capabilities of human cerebellum.

Returning back to FIG. 4, at step 405, the extracted features of the cardiac signal are provided to the classification module. In one configuration, the classifier, can include, but is not limited to, an echo state network. The echo-state network segments and for classifying multiple sound sources associated with the cardiac signal, for instance, those extracted features related to atrial valve opening/closing, semilunar valve opening/closing, blood flow (acceleration/deceleration), respiratory sounds, etc. The classification module, by way of the processor 310, implements the biologically inspired separation of heart sounds as a function of sound patterns modeled from mechanical and physiological processes of the heart determined through phsychoacoustic analysis. It classifies heart sound patterns in accordance with biologically based signal processing models of the auditory cortex and cerebellum noted above. As one example, it models the mechanical event vibrations of the heart sounds characterized from vibratory movement of inner hair cells along the basilar membrane. It retrieves from memory module 306, pre-stored (or pre-learned) mechanical event feature patterns associated with the vibrations of blood, heart walls and valves corresponding to mechanical events of cardiac cycles, and compares the pre-stored mechanical event feature patterns to extracted features of the heart sounds captured by the sensor array. It classifies heart sounds for both congenital and acquired heart diseases. As one example, the pre-stored models are derived from phsychoacoustic analysis of known regular and irregular heart sounds.

In another embodiment, the echo-state network emulates the sophisticated computational units and processes of the auditory cortex and cerebellum in the human brain. The cerebellum is a richly connected network of neurons, each of which may respond differently to the same input, yet is fairly homogenous. The outputs of neurons and synapses in the auditory cortex depend in diverse ways on the recent history of their inputs and the brain carries out real-time computations on time-varying input streams that require integration of information over time. These time-varying connections and associated history are emulated in the echo-state network as interconnected states and transitions. The states model both vibrations from infrasound and acoustically transmitted audible sound. For example, one state may be associated with the extracted features of infrasound specific to opening and closing of a valve, while another state, or interconnected group of states, is associated with audible sounds, and more specifically, acoustic cues or patterns characteristic to regular and irregular heart functions associated with these mechanical events.

This interconnected network provides a human factor analysis mapping between the infrasound and acoustically transmitted audible sounds. The echo-state network in this configuration, by way of the processor 310 and memory 306, realizes a neurophysiological classifier specific to heart sounds. It models the human cerebellum as a recurrently connected reservoir of neurons. The reservoir can have multiple read-outs, for classifying detected sound patterns associated with mechanical heart function events. One example, for classifying multiple sound sources associated with a sound signal, such as a cardiac signal, can be realized with an auditory scene analyzer. The disclosure herein, for such purpose, claims priority benefit to Provisional Patent Application No. 61/463,069 filed on Feb. 11, 2011, entitled “Method and System of an Acoustic Scene Analyzer with Body Sounds”, the entire contents of which are hereby incorporated by reference.

FIG. 7 illustrates a Time-Frequency analysis 700 of heart sounds in accordance with one embodiment. The analysis provides a two-dimensional image over time, analogous to a spectrogram configured along a psychoacoustic frequency and time scale. Classifier outputs of the detected heart sounds are shown: S1, S2, S3 and murmurs are referred according to the standard definitions as follows. These are the first heart sound (S1) and second heart sound (S2), produced by the closing of the AV valves and semilunar valves respectively. In addition to these normal sounds, a variety of other sounds may be present including heart murmurs M. A third heart sound (S3) called a protodiastolic or ventricular gallop. A fourth heart sound (S4) called a presystolic or atrial gallop. The heart sound heard on the chest cavity is a composite sound comprising of these individual heart sounds.

Returning back to the method 400 in FIG. 4, at step 407, the classification output, performed in accordance with biologically based signal processing models of the auditory cortex and cerebellum, is displayed. This can be provided through the display (e.g., LCD) module 304 or through a remote monitor over the network 190 communicatively coupled to the portable device 102 through the transceiver 302. As one example, shown in FIG. 1, the portable device display can overlay the classification output (e.g., S1, S2, etc.) with the captured cardiac signal, or other plots of the heart beat signal. The classification output can further indicate a type of heart condition, for example, whether a heart murmur is innocent or pathological, the type of innocent murmur, the type of pathological murmur, or other cardiovascular classification. The classifier upon detecting can report normal and abnormal conditions of the cardiovascular system for both congenital and acquired heart diseases. It can furthermore diagnose and monitor cardiovascular condition based on the classification of the heart sound patterns.

It will be apparent to those skilled in the art that various modifications may be made in the present invention, without departing from the spirit or scope of the invention. Thus, it is intended that the present invention cover the modifications and variations of this invention provided they come within the scope of the method and system described and their equivalents.

For example, variations of the exemplary embodiments describe a portable solution that can be used in the prevention and treatment of heart failure, prenatal and postnatal detection of congenital heart defects, military and athletic screening assessment and monitoring of cardiovascular status. This information can be reported locally to the immediate user, or by way of the network 120 and location unit 308, the portable cardio-acoustic device can report a user's location for scenarios requiring critical attention.

The exemplary embodiments provide a novel method of capturing sound below hearing threshold on the embedded platform. The novel method allows the exemplary embodiments to operate in real-life situations and provide for accurate diagnosis of cardiovascular condition by non-clinicians.

The exemplary embodiments provide a novel method of feature extraction from the infrasound and normal hearing range of heart sounds for greater fidelity. The novel method allows the exemplary embodiments to operate in real-life situations and provide for accurate diagnosis by non-clinicians.

The exemplary embodiments develop a novel method of automated measurement, assessment and classification of heart sounds on the embedded platform. The novel method allows the exemplary embodiments to operate in real-life situations and provide for accurate diagnosis by non-clinicians.

The exemplary embodiments provide a neuro-physiological classifier optimized for heart sounds on a small embedded platform for classifying murmur as innocent, pathological or specific cardiovascular condition, using heart sounds.

As an example, the embedded platform can be any smart processing platform with digital signal processing capabilities, application processor, data storage, display, input modality touch-screen or keypad, microphones, speaker, Bluetooth, and connection to the internet via WAN, Wi-Fi, Ethernet or USB.

Where applicable, the present embodiments of the invention can be realized in hardware, software or a combination of hardware and software. Any kind of computer system or other apparatus adapted for carrying out the methods described herein are suitable. A typical combination of hardware and software can be a portable communications device with a computer program that, when being loaded and executed, can control the portable communications device such that it carries out the methods described herein. Portions of the present method and system may also be embedded in a computer program product, which comprises all the features enabling the implementation of the methods described herein and which when loaded in a computer system, is able to carry out these methods.

While the preferred embodiments of the invention have been illustrated and described, it will be clear that the embodiments of the invention is not so limited. Numerous modifications, changes, variations, substitutions and equivalents will occur to those skilled in the art without departing from the spirit and scope of the present embodiments of the invention as defined by the appended claims.

What is claimed is:

1. A portable cardio-acoustic device, comprising:
a display with user input;
a sensor array to capture from heart sounds, both vibrations from infrasound and acoustically transmitted audible sound;
a processor coupled to the display and sensor array to:
extract salient features from a captured heart sounds in accordance with human factor analysis;
separate heart sounds as a function of sound patterns modeled from mechanical and physiological processes of the heart determined through phsychoacoustic analysis;
classify heart sound patterns in accordance with biologically based signal processing models of the auditory cortex and cerebellum; and
diagnose and monitor cardiovascular condition based on the classification of the heart sound patterns,
a power supply to provide power to electronic components of the portable cardio-acoustic device.

2. The portable cardio-acoustic device of claim 1, wherein the sensor array comprises non-contact microphones,piezoelectric film sensor, or accelerometer contact microphones that do not capture environmental noise to:
provide a unique sound or vibration pick-up with buffered output ideal for detecting body sounds, and
minimize external acoustic noise while offering extremely high sensitivity to vibration, with high sensitivity in infrasound region below 100 Hz and in audible regions above 100 Hz.

3. The portable cardio-acoustic device of claim 2, further comprising a memory, wherein the processor:
retrieves from the memory, pre-learned mechanical event feature patterns associated with the vibrations of blood, heart walls and valves corresponding to mechanical events of cardiac cycles; and
compares the pre-stored mechanical event feature patterns to extracted features of the heart sounds captured by the sensor array.

4. The portable cardio-acoustic device of claim 3, wherein the processor, prior to generating the extracted features, applies a human factor critical band filter-bank to the infrasound and acoustically transmitted audible sound to increase resolution below 100 Hz and enhance sensitivity to the lower frequency regions specific to the heart sounds.

5. The portable cardio-acoustic device of claim 1, wherein human factor analysis comprises:
enhancing sensitivity in a low frequency infrasound range of the heart sounds; and
identifying variations of the cardiac signal in the infrasound range according to classified heart disease indicators.

6. The portable cardio-acoustic device of claim 1, wherein human factor analysis comprises:
comparing sound patters against pre-determined models of the mechanical and physiological processes of heart sounds determined through phsychoacoustic analysis; and
classifying heart sounds including a murmur for both congenital and acquired heart diseases,
classifying if the murmur detected in the heart sounds is innocent or pathological and a type of innocent murmur,
wherein the pre-stored models are derived from phsychoacoustic analysis of known regular and irregular heart sounds, and including identifying the type of innocent murmur and type of pathological murmur or other cardiovascular classification.

7. The portable cardio-acoustic device of claim 1, wherein the portable cardio-acoustic device monitors and reports a health status responsive to the classification of the heart sounds.

8. The portable cardio-acoustic device of claim 1, further comprising detecting and reporting normal and abnormal conditions of the cardiovascular system for both congenital and acquired heart diseases.

9. The portable cardio-acoustic device of claim 1, further comprising detecting and reporting normal and abnormal conditions of the cardiovascular condition.

10. A method for assessing cardiovascular condition by way of a portable cardio-acoustic device, the method comprising the steps of:
capturing from heart sounds, both vibrations from infrasound and acoustically transmitted audible sound;
extracting salient features from a captured cardiac signal of the heart sounds in accordance with human factor analysis;
separating heart sounds as a function of sound patterns modeled from mechanical and physiological processes of the heart determined through phsychoacoustic analysis;
classifying the captured cardiac signals in accordance with biologically based signal processing models of the auditory cortex and cerebellum; and
diagnosing and monitoring cardiovascular condition based on the classification of the heart sound patterns.

11. The method of claim 10, further comprising the steps of:
retrieving from the memory, pre-stored mechanical event feature patterns associated with the vibrations triggered by the acceleration and deceleration of blood due to abrupt mechanical events of cardiac cycles; and
comparing the pre-stored mechanical event feature patterns to extracted features of the heart sounds captured by the sensor array.

12. The method of claim 11, further comprising the steps of:
applying a human factor critical band filter-bank to the infrasound and acoustically transmitted audible sound to increase resolution below 100 Hz and enhance sensitivity to the lower frequency regions specific to the heart sounds.

13. The method of claim 10, wherein the feature extraction in the infrasound region is modeled on auditory signal processes of the human cochlea, which convert pressure changes of the ear drum to vibratory movement of a basilar membrane, to match human audibility in the 100 to 10 KHz bandwidth.

14. The method of claim 13, wherein the modeling of the mechanical event vibrations of the heart sounds are characterized from vibratory movement of inner hair cells along the basilar membrane.

15. The method of claim 11, wherein the frequency extraction encodes a time-frequency sound signal decomposition according to frequency perception derived from inner hair cells activation responses grouped in critical bands along the basilar membrane.

16. The method of claim 10, non-linearily amplify the heart sounds in the infrasound bandwidth below hearing threshold in accordance with a psychoacoustic compression to compensate for the biological representation of loudness, pitch and timbre of human hearing.

17. The method of claim 10, non-linearily frequency shift the cardiac signal in the infrasound bandwidth to the audible bandwidth in accordance with a human hearing frequency scale to compensate for the biological representation of loudness, pitch and timbre of human hearing thereby permitting audibility of the heart sound above human hearing sensitivity threshold.

18. A system for assessing cardiovascular condition, comprising:
a portable cardio-acoustic device, having:
a display with user input;
a sensor array to capture from heart sounds, both vibrations from infrasound and acoustically transmitted audible sound;
a processor coupled to the display and sensor array to:
extract salient features from a captured heart sounds in accordance with human factor analysis;
separate heart sounds as a function of sound patterns modeled from mechanical and physiological processes of the heart determined through phsychoacoustic analysis, and
a power supply to provide power to electronic components of the portable cardio-acoustic device,
and,
a remote server communicatively coupled to the portable cardio-acoustic device, to
classify heart sound patterns in accordance with biologically based signal processing models of the auditory cortex and cerebellum; and
diagnose and monitor cardiovascular condition based on the classification of the heart sound patterns.

19. The system of claim 18, wherein the remote server
compares sound patters against pre-determined models of the mechanical and physiological processes of heart sounds stored in a local database determined through phsychoacoustic analysis; and
classifies if a murmur detected in the heart sounds from the sound patternsis innocent or pathological and a type of innocent murmur,
wherein the pre-stored models are derived from phsychoacoustic analysis of known regular and irregular heart sounds, and including identifying the type of innocent murmur and type of pathological murmur or other cardiovascular classification.

20. The system of claim 19, wherein the remote server responds to a software application executing on a mobile device requesting classification of the sound patterns and visually presents a psychoacoustic interpretation of sound patterns for on-line display and on the mobile device.
